(19) United States
(12) Patent Application Publication
(10) Pub. No.: US 2020/0166499 A1
(43) Pub. Date: May 28, 2020

(54) THREE-DIMENSIONAL CELL CULTURE MODEL OF THE HUMAN SWEAT GLAND FOR THE ANALYSIS OF STRESS-ASSOCIATED SWEATING PROCESSES

(71) Applicant: Henkel AG & Co. KGaA, Duesseldorf (DE)

(72) Inventors: THOMAS WELSS, Duesseldorf (DE); BERNHARD BANOWSKI, Duesseldorf (DE); JESSICA WELZEL, Duesseldorf (DE); SABINE GRUEDL, Erkelenz (DE)

(73) Assignee: Henkel AG & Co. KGaA, Duesseldorf (DE)

(21) Appl. No.: 16/584,350

(22) Filed: Sep. 26, 2019

(30) Foreign Application Priority Data
Nov. 26, 2018 (DE) 10 2018 129 793.9

(51) Int. Cl. G01N 33/50; C12N 5/071; C12N 5/00; G01N 33/52

(52) U.S. Cl. CPC G01N 33/5082; C12N 5/0633; G01N 33/52; C12N 5/0018; C12N 5/0062; G01N 33/5044

(57) ABSTRACT

A method for the in-vitro investigation of sweating processes, in which a three-dimensional sweat gland equivalent is stimulated adrenergically, and the reaction of the three-dimensional sweat gland equivalent is determined by measuring the cyclic adenosine monophosphate (cAMP) content.

### CROSS-REFERENCE TO RELATED APPLICATION

This application claims priority to German Patent Application No. 10 2018 129 793.9, filed Nov. 26, 2018, which is incorporated herein by reference in its entirety.

### TECHNICAL FIELD

The present disclosure relates to a method for the in-vitro investigation of sweating processes, in which a three-dimensional sweat gland equivalent is provided, the three-dimensional sweat gland equivalent is stimulated adrenergically, and the reaction of the three-dimensional sweat gland equivalent is determined.

### BACKGROUND

Washing, cleaning and care of an individual's body is a basic human necessity and modern industry is constantly looking out for ways to do justice to these human necessities in manifold manners. What is particularly important for daily hygiene is the sustained removal or at least reduction of body odor and armpit wetness. Customers want antiperspirants which effectively prevent the negative effects of emotional sweating on their quality of life. Protecting against body odor and armpit wetness with cosmetic products is an important part of daily hygiene. It provides the user with security and self-confidence—especially in people who are under psychological pressure due to excessive sweat production.

Armpit wetness and body odor arise from the secretions from eccrine and apocrine sweat glands in the human armpit. While the eccrine glands are primarily responsible for the regulation of heat in the body and are responsible for the occurrence of armpit wetness, the apocrine glands exude a viscous secretion in reaction to stress, from which an unpleasant body odor arises when it undergoes bacterial decomposition.

Initial research studies on native eccrine and apocrine sweat glands were carried out as early as the beginning of the 20th century in order to classify them into the group of skin appendages belonging to the exocrine gland group. Thereafter, sweat glands were divided into apocrine and eccrine sweat glands as well as a hybrid of apocrine and eccrine sweat glands (also known as apoeccrine sweat glands). The forms mentioned above can be distinguished on the basis of their morphological and characteristic features.

The eccrine sweat gland, in particular the human eccrine sweat gland, belongs to the unbranched coiled tubular glands and can be divided into the secretory base (also known as the coil), the dermal excretory duct (also known as the duct) and the epidermal excretory duct (also known as the acrosyringium). The cells present in these sections of the gland have different purposes and functions such as, for example, secretion in the coil, reabsorption of ions in the duct, as well as exuding the secretion, in particular sweat, onto the surrounding skin through the acrosyringium. The eccrine sweat glands are primarily stimulated by the neurotransmitter acetylcholine (ACh). In respect of preventing armpit wetness and/or body odor, reducing and/or preventing secretions from eccrine and/or apocrine sweat glands is desirable. This may be carried out, for example, by obstructing the excretory ducts of eccrine sweat glands by what are known as plugs. In the prior art, sweat-inhibiting aluminum and/or aluminium-zirconium salts are used in this regard; however, consumers are now skeptical about them.

Furthermore, antibacterial agents are used in the prior art which prevent the bacterial decomposition of sweat. However, such agents can have a negative influence on the natural microflora of the skin under the armpit. Thus, it would be apposite to provide cosmetic agents which were capable of reliably preventing armpit wetness and/or body odor and which contained neither the aluminum and/or aluminum-zirconium salts nor the antibacterial agents used in the prior art. Individual, unverified studies associate these aluminum compounds with diseases. The unsettled consumer wants “aluminum-free” products. Although there is so far no proof in this regard and the Bundesamt für Risikobewertung [German Federal Institute for Risk Assessment] (BfR) has no reports of any risks to users during normal use, the media and consumers are on high alert in this regard.

One possibility for providing such agents arises from using substances which effectively inhibit the stimulation of the sweat glands and thus reduce or prevent the secretion of sweat. In order to be able to identify such substances, in-vivo tests with trial participants can be carried out. However, such tests are expensive and are not suitable for high-throughput screening methods. On the other hand, in-vitro tests may be carried out using cell models of sweat glands on which the influence of test substances on the stimulation of the sweat glands can be investigated.

So that the in-vitro test results can be properly applicable to the in-vivo situation, the cell model employed must emulate the in-vivo situation as closely as possible. For this, three-dimensional cell models are necessary, because the known two-dimensional models of the prior art are not physiologically close enough to native sweat glands, and are therefore poor imitators of the in-vivo situation. Moreover, an elucidation of the sweat secretion mechanism is required. This is because only in this manner can what are known as biological targets be identified, in particular proteins produced by the sweat gland cells, which are influenced by the test substances to produce less sweat. Possible biological targets which could be linked to sweat production are ion channels and/or water channels and/or receptors for signal transduction which control the secretion of sweat.

Furthermore, cell models of sweat glands may be used, on which the influence of test substances on the stimulation of the sweat glands can be investigated. Such models have to simulate the in-vivo situation as closely as possible, and they must be capable of being standardized and must be inexpensive, as well as be suitable for use with high-throughput screening methods. An example of a known three-dimensional sweat gland model from the prior art is described by Li (Li et. al.; “Matrigel basement membrane matrix induces eccrine sweat gland cells to reconstitute sweat gland-like structures in nude mice”; Experimental Cell Research, 2015, 332, pages 67 to 77). In order to produce that sweat gland model, initially, primary sweat gland cells are cultured with growth factors in a gel-like substance (Matrigel®) and then implanted in the backs of live mice. After implantation, spherical structures are formed which express sweat gland-specific marker proteins. Because mice are required to construct the differentiated structures, this model cannot be used in cosmetic and pharmaceutical research, where the use of research animals is prohibited. Finally, the use of full skin models, which also contain sweat glands along with a dermal equivalent and an epidermis, is also known. By punching specimens from native adult human skin, models are obtained which contain all of the skin appendages, which encompasses sweat glands, in this region of the skin. Such models are also known as ex-vivo full skin models.

Thus, there is still a need for in-vitro methods with the aid of which biological targets which are responsible for an increased production of sweat can be identified and analyzed. After the identification and analysis of such targets; an investigation of the influence of various test substances on these targets is preferably carried out. In-vitro methods of this type should be capable of being standardized, should be inexpensive and should be rapid to carry out, so that the influence of the test substances on the biological targets can be determined using high throughput screening methods.

### BRIEF SUMMARY

In an embodiment, a method is provided for the in-vitro investigation of sweating processes, in which a three-dimensional sweat gland equivalent is provided, the three-dimensional sweat gland equivalent is stimulated adrenergically, and the reaction of the three-dimensional sweat gland equivalent is determined by measuring the cyclic adenosine monophosphate (cAMP) content. In various embodiments, the three-dimensional sweat gland equivalent is produced by means of a method that comprises the following steps:

a) providing isolated sweat glands,

b) providing a cell preparation of primary sweat gland cells from the sweat glands isolated in step a) of the method,

c) applying the cell preparation provided in step b) of the method to a substrate,

d) culturing the cell preparation applied in step c) of the method,

e) isolating the three-dimensional sweat gland equivalent obtained in step d) of the method.

In another embodiment, a method is provided for screening active substances for antiperspirant activity, wherein a three-dimensional sweat gland equivalent is provided, the three-dimensional sweat gland equivalent is stimulated adrenergically, the three-dimensional sweat gland equivalent is brought into contact with an active substance to be investigated before, during and/or after adrenergic stimulation, and the reaction of the three-dimensional sweat gland equivalent is determined by measuring the cyclic adenosine monophosphate (cAMP) content, wherein a reduction in cAMP content compared to a control that has not been brought into contact with an active substance is indicative of antiperspirant activity.

In any of these embodiments, the three-dimensional sweat gland equivalent is stimulated by an adrenergic drug, which can be an adrenergic receptor agonist and/or an adrenergic reuptake inhibitor, for example adrenaline.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Thus, the objective of the present disclosure is to provide an in-vitro method for the analysis of sweating processes that is capable of being standardized, is inexpensive and which can also be carried out rapidly and the results therefrom should be applicable to the in-vivo situation. By means of the method, substances which constitute potentially effective antiperspirants should be able to be identified.

The objective is achieved by means of a method in accordance with claim 1. Thus, the subject matter of the present disclosure is a method for the in-vitro investigation of sweating processes, in which a three-dimensional sweat gland equivalent is provided, the three-dimensional sweat gland equivalent is stimulated adrenergically, and the reaction of the three-dimensional sweat gland equivalent is determined by measuring the cyclic adenosine monophosphate (cAMP) content.

It has been discovered that an in-vitro investigation of sweating processes may be carried out by detecting the reaction of a three-dimensional sweat gland equivalent by measuring the cyclic adenosine monophosphate content.

The first step of the method concerns the preparation of a three-dimensional sweat gland equivalent.

The term “three-dimensional sweat gland equivalent” as contemplated herein should be understood to mean a cell model formed from sweat gland cells, which extends in all three directions in space and in which the cells exhibit a similar function, in particular an identical function, to the cells of a native sweat gland. The production of three-dimensional sweat gland equivalents is known in the prior art. By introducing three-dimensional sweat gland equivalents into a dermis and/or epidermis equivalent of a skin equivalent, models can be obtained in which the cell-cell interactions between the various cell types closely correspond to those in native skin. The production is carried out exclusively by means of in-vitro methods and allows for a high level of standardization as well as inexpensive production of the skin equivalents. Thus, this skin equivalent is particularly suitable for use in high throughput screening methods for testing sweat-inhibiting substances for use in cosmetics and in pharmaceuticals. In a subsequent step of the method, the three-dimensional sweat gland equivalent that is provided is stimulated adrenergically. The stimulation is intended to simulate the human stress factor. In principle, any stimulants which stimulate adrenergic receptors may be considered. Now, the three-dimensional sweat gland equivalent can be exposed to an active substance.

If the stimulated three-dimensional sweat gland equivalent is exposed to an active substance, then this could result in a reaction of the three-dimensional sweat gland equivalent. A step of the method in the method as contemplated herein may comprise bringing the three-dimensional sweat gland equivalent into contact with a substance to be investigated. Depending on the type of substance to be investigated, this could result in a desired inhibition within the adrenergic signal pathway. The extent of the inhibition of the adrenergic signal pathway allows conclusions to be drawn as regards the potential of the active substance to be investigated to act as an antiperspirant. The extent of the inhibition of the adrenergic signal pathway is determined in the present disclosure by measuring the cyclic adenosine monophosphate (cAMP) content. Cyclic adenosine monophosphate (cAMP) is a molecule which is biochemically derived from adenosine triphosphate (ATP), which acts as what is known as a “second messenger” in the cellular transmission of nerve impulses. Finally, the cAMP content serves as a quantitative measure of the antiperspirant action of a potential active substance. In the last step of the method as contemplated herein, therefore, the reaction of the three-dimensional sweat gland equivalent to an active substance is determined, in fact by measurement of the cAMP content. The more active the cells in the cell complex are, the higher is the cAMP value. The cAMP content should be reduced by the active substance to be investigated despite adrenergic stimulation. In accordance with a preferred embodiment of the present disclosure, the three-dimensional sweat gland equivalent comprises from about 500 to about 500000 sweat gland cells. In embodiments, the three-dimensional sweat gland equivalent has a diameter of from about 100 μm to about 6000 μm, from about 150 μm to about 4000 μm, or from about 200 μm to about 3000 μm. The diameter of the spherical sweat gland equivalents as contemplated herein may, for example, be determined by means of microscopic measurement using the “CellSens” software. Three-dimensional sweat gland equivalents with the above properties may be produced using a method as described below. In accordance with a preferred embodiment of the present disclosure, the three-dimensional sweat gland equivalent is produced by means of a method which comprises the following steps:

a) providing isolated sweat glands,
b) providing a cell preparation of primary sweat gland cells from the sweat glands isolated in step a) of the method,
c) applying the cell preparation provided in step b) of the method to a substrate,
d) culturing the cell preparation applied in step c) of the method,
e) isolating the three-dimensional sweat gland equivalent obtained in step d) of the method.

The term “sweat glands isolated in step a) of the method” should be understood to mean non-cultured sweat glands which may be obtained from skin biopsies or the like and which have been removed from their natural environment. These isolated sweat glands are subsequently prepared, cultured and isolated in order to obtain a cell culture. The term “cell preparation of primary sweat gland cells used in step b) of the method” should be understood to mean a suspension of sweat gland cells of the cultured isolated sweat gland in a solution, in particular in a nutrient medium. In this connection, preferably, the isolation of the native sweat glands is preferably carried out by enzymatic digestion of the human skin using a mixture of from about 2 to about 3 mg/mL of collagenase II and from about 0.1 to about 0.2 mg/mL of thermolysin for from about 3 to about 6 hours at from about 35° C. to about 40° C., in particular at about 37° C. The human skin used in this regard is preferably a skin biopsy which is generated, for example during cosmetic surgery, as a waste product. Furthermore, preferably, a hanging drop preparation may be carried out, which should be understood to mean a preparation in which the cell preparation from step b) of the method is not placed on the surface of a culture plate or flask but is in the form of a droplet suspended freely from a surface or suspended freely between two surfaces.

In step b) of the method as contemplated herein, a cell preparation of primary sweat gland cells is provided from the isolated, preferably native sweat glands. This cell preparation may, for example, be produced after preparing the isolated, preferably native sweat glands, dissociating the monolayer cultures which have grown from the sweat glands and preparing these dissociated primary sweat gland cells by suspension in a suitable medium and adjustment to the required cell count. Thus, embodiments which are preferred as contemplated herein are exemplified in that the cell preparation of primary sweat gland cells in step b) of the method is produced by preparation of the isolated sweat glands in step a) of the present disclosure, dissociating the sweat gland cells, in particular by careful trypsinization, culturing these detached sweat gland cells in monolayer cultures, suspending the cultured primary sweat gland cells in a nutrient medium as well as adjusting the cell count.

In accordance with the preferred embodiment, following provision of the cell preparation of primary sweat gland cells, the cell preparation is applied to a substrate. In other words, in step c) of the method, the cell preparation of primary sweat gland cells provided in step b) of the method is applied to a surface, preferably to a polymer surface. In order to obtain a sufficient level of standardization, prior to culturing the cell preparation of primary sweat gland cells, a specific cell count on the polymer surface must be established. In order to allow the formation of different characteristics as well as the differentiation of the primary sweat gland cells into different functionalities in the three-dimensional sweat gland equivalent, the cell preparation applied to the surface, preferably to the polymer surface in step c) of the method, contains specific cell counts of primary sweat gland cells. The use of precisely defined cell counts also enables the sweat gland equivalents to be standardized, because all equivalents have similar, in particular identical cell counts and thus results that diverge from each other or a complicated standardization can be avoided. Preferred embodiments of this aspect of the present disclosure are thus exemplified in that in step c) of the method, the cell count for the primary sweat gland cells in the cell preparation is from about 5000 to about 230000 cells per square centimeter of polymer surface, from about 5000 to about 200000 cells per square centimeter of polymer surface, from about 5000 to about 170000 per square centimeter of polymer surface, from about 5000 to about 140000 cells per square centimeter of polymer surface, or from about 5000 to about 132000 cells per square centimeter of polymer surface.

The cell count of the primary fibroblasts can, for example, be determined using a conventional counting chamber as well as trypan blue. To this end, a trypan blue solution is added to an undiluted suspension of cultured primary fibroblasts and the number of cells in the appropriate corner squares is determined. The arithmetic mean is produced from these values. Considering the volume of the counting chamber, the dilution factor and the chamber factor, the cell count per mL or μL is determined from this mean value. The cell count used in method step c) in this regard is in respect of the number of live cells (live count). By using an appropriate volume, the required cell count may be applied to the polymer surface. In the context of the present disclosure, preferably, the cell preparation of primary sweat gland cells used in step c) of the method has a specific volume. This makes culturing of this cell preparation on the substrate easier. Thus, as contemplated herein, it is preferable if in step c) of the method, the cell preparation of primary sweat gland cells to be applied to the cell surface has a volume of from about 400 to about 4000 μL, from about 450 to about 3000 μL, from about 480 to about 2500 μL, or of from about 490 to about 2.100 μL. Application of the pre-specified volume onto the substrate, in particular onto the polymer surface, ensures sufficient growth of the primary sweat gland cells in this cell preparation.

Culturing of the cell preparation of primary sweat gland cells on the substrate, in particular on the special polymer surface, is carried out in step d) of the method as contemplated herein.

The polymer surface prevents the adhesion of cells onto this surface and therefore supports the formation of the three-dimensional sweat gland equivalent.

A preferred embodiment of the present disclosure is therefore exemplified in that the polymer surface is formed by at least one polymer selected from the group formed by polyvinyl alcohols, polyethylene glycols, poly(2-hydroxyethyl)methacrylates, dextrans, carboxymethylcelluloses, agaroses, starches, celluloses, polyacrylamides, esters of alginic acids and/or hyaluronic acids and/or polyacrylates and/or pectins, as well as mixtures thereof. Examples of polymer surfaces of this type are commercially available from Corning Incorporated as Ultra Low Attachment Cell Culture Products with covalently bonded hydrogel surfaces.

It has been shown to be advantageous for the cell preparation with the primary sweat gland cells in step d) of the method to be cultured for a specific period of time under specific conditions on the substrate, in particular on the polymer surface. Thus, preferably, culture of the cell preparation in step d) of the method is carried out for a period of from about 1 to about 25 days, or from about 2 to about 7 days, at a temperature of from about 36° C. to about 38° C. and with a CO₂ content of about 5% by weight with respect to the total weight of the atmosphere employed for culture.

In the context of the present disclosure, it may also be provided that during culture of the primary sweat gland cells in step d) of the method, the nutrient medium used for applying the cell preparation is replaced with fresh nutrient medium. The term “fresh nutrient medium” herein should be understood to mean a nutrient medium which contains no cells whatsoever. The proportion by volume in this case is with respect to the total volume of the cell preparation produced in step h) of the method. Thus, as contemplated herein, it is advantageous if, during the period of culture in step d) of the method, in particular after from about 1 to about 3 days, the nutrient medium for the cell preparation is replaced with fresh nutrient medium.

In this connection, preferably, a specific nutrient medium is used as fresh nutrient medium for changing out the volume. In the context of the present disclosure, therefore, advantageously, a mixture formed by DMEM and Ham's F12 is used as the fresh nutrient medium in a weight ratio of about 3:1, additionally containing about 10% by weight of fetal calf serum (FCS) with respect to the total weight of the mixture. In step e) of the method in the method as contemplated herein, isolation of the three-dimensional sweat gland equivalent produced is carried out by removing the nutrient medium. Thus, preferred embodiments of the present disclosure are exemplified in that the isolation of the three-dimensional sweat gland equivalent is carried out in step e) of the method by removing the nutrient medium.

Particularly preferably, the three-dimensional sweat gland equivalent is an equivalent of the human eccrine and/or apocrine sweat gland. Thus, preferred embodiments of the present disclosure are exemplified in that the three-dimensional sweat gland equivalent is a three-dimensional sweat gland equivalent of the human eccrine and/or apocrine sweat gland. In particular, in step a) of the production method, they are eccrine sweat glands.

The sweat gland equivalents obtained in accordance with the preferred production method are particularly suitable for the identification of sweat-inhibiting active substances for in-vivo application in human beings,

The three-dimensional sweat gland equivalents as contemplated herein have a high level of standardization and availability, like isolated sweat glands, and resemble the in-vivo situation more closely than one-dimensional and two-dimensional sweat gland models. Furthermore, these equivalents constitute an inexpensive alternative to in-vivo studies in human beings, because the sweat-inhibiting action of test substances can be investigated by means of the equivalents, for example by comparing gene expression or protein expression during stimulation with acetylcholine (Ach) in the presence and absence of a specific test substance. The three-dimensional sweat gland equivalents as contemplated herein simulate the sweat gland in-vivo both as regards its structure and also as regards its histological composition, so that the information obtained with these equivalents is applicable to human beings and can also be compared with data for compounds which have already been tested in-vivo. In accordance with a further preferred embodiment, the three-dimensional sweat gland equivalent is in an aqueous medium or is dispersed in water. The advantage associated with this feature is that active substances to be investigated that are soluble in water, which could act as antiperspirants, can be investigated as regards their antiperspirant potential.

In the context of the present disclosure, preferably, the sweat gland equivalents which are produced as contemplated herein are free from matrix compounds and/or supports. The term “matrix compounds” as used herein should be understood to mean compounds which are capable of forming spatial networks. However, this does not include the substances which are produced and excreted by the cells per se that are capable of forming spatial networks. Furthermore, the term “supports” as used in the context of the present disclosure should be understood to mean self-supporting substances that could act as a substrate or scaffold for the sweat gland cells. In accordance with a preferred embodiment of the present disclosure, the three-dimensional sweat gland equivalent is free from matrix compounds and/or supports, in particular free from matrix compounds and supports.

The term “free from” as contemplated herein should be understood to mean that the three-dimensional sweat gland equivalents contain less than about 1% by weight, with respect to the total weight of the three-dimensional sweat gland equivalent, of matrix compounds and/or supports. Thus, in the context of the present disclosure, the three-dimensional sweat gland equivalents contain from 0 to about 1% by weight, from 0 to about 0.5% by weight, from 0 to about 0.2% by weight, or 0% by weight of matrix compounds and supports, with respect to the total weight of the three-dimensional sweat gland equivalent. In this regard, it is particularly advantageous for the three-dimensional sweat gland equivalents to be free from specific matrix compounds and supports. Thus, preferably, the three-dimensional sweat gland equivalent does not contain any matrix compounds and/or supports which are selected from the group formed by collagens, in particular collagen type I and/or type III and/or type IV, scleroproteins, gelatins, chitosans, glucosamines, glucosaminoglucans (GAG), heparin sulfate proteoglucans, sulfated glycoproteins, growth factors, cross-linked polysaccharides, cross-linked polypeptides, as well as mixtures thereof.

In accordance with a preferred embodiment, the primary sweat gland cells provided in step b) of the method comprise at least one cell type selected from the group formed by (i) coil cells, in particular clear cells, dark cells, as well as myoepithelial cells, (ii) duct cells, as well as (iii) mixtures thereof. Special coil cells are responsible for the formation of primary sweat. The selected cells of the coil are therefore advantageously used as the basis for the production of three-dimensional sweat gland equivalents, which are to be used in a method for the investigation of sweating processes. The duct constitutes the excretory duct for the sweat cell. Duct cells have the function of ion resorption and therefore have an influence on sweating processes and in particular on the development of odors, because the cells influence the salt content. Furthermore, adrenergic receptors have been found on the cell types which are preferably employed.

In accordance with a preferred embodiment of the method as contemplated herein, the three-dimensional sweat gland equivalent is stimulated, wherein adrenaline, preferably an aqueous solution comprising adrenaline, is added to the three-dimensional sweat gland equivalent. The stimulation constitutes the simulation of stress. By contact with adrenaline as a preferred stimulator, the sweat gland equivalent should be stimulated in the same way that stress stimulates the natural sweat glands in the human body. In this regard, adrenaline is brought into contact with the aqueous solution in an aqueous suspension comprising the three-dimensional sweat gland equivalent.

The reaction of the sweat gland equivalent is registered by detecting the presence of cAMP, whereupon its concentration is measured. Specifically, in accordance with a preferred embodiment of the present disclosure, the concentration of the cAMP is measured colorimetrically by means of a color reaction. The more active are the cells in the cell complex, the higher is the cAMP value. Bringing active substances into contact with the three-dimensional sweat gland equivalent should reduce the cAMP content in spite of adrenergic stimulation. The lower the cAMP content is after contacting, the better is the active substance to be investigated. In accordance with a preferred method, the reaction of the three-dimensional sweat gland equivalent to an active substance that is envisaged as reducing the sweating process is determined on the three-dimensional sweat gland equivalent.

In accordance with a further preferred embodiment, the reaction of the three-dimensional sweat gland equivalent to the active substance is determined, wherein the active substance is added to the three-dimensional sweat gland equivalent and in fact preferably before the three-dimensional sweat gland equivalent is stimulated, or at the same time as the step of the method in which the three-dimensional sweat gland equivalent is stimulated, or after the three-dimensional sweat gland equivalent has been stimulated. By using these three alternatives, the mechanism of the reaction of the three-dimensional sweat gland equivalent can be investigated especially well. More particularly preferred cosmetic compositions as contemplated herein comprise at least one of the following embodiments A) to E):

### A)

A method for the in-vitro investigation of sweating processes, in which

a three-dimensional sweat gland equivalent is provided,

the three-dimensional sweat gland equivalent is stimulated adrenergically,

the three-dimensional sweat gland equivalent is brought into contact with an active substance to be investigated, and

the reaction of the three-dimensional sweat gland equivalent is determined by measuring the cyclic adenosine monophosphate (cAMP) content.

### B)

A method for the in-vitro investigation of sweating processes, in which

a three-dimensional sweat gland equivalent is provided,

the three-dimensional sweat gland equivalent is stimulated adrenergically,

the three-dimensional sweat gland equivalent is brought into contact with an active substance to be investigated, and

the reaction of the three-dimensional sweat gland equivalent is determined by measuring the cyclic adenosine monophosphate (cAMP) content, wherein the three-dimensional sweat gland equivalent is produced by means of a method that comprises the following steps:

a) providing isolated sweat glands,
b) providing a cell preparation of primary sweat gland cells from the sweat glands isolated in step a) of the method,
c) applying the cell preparation provided in step b) of the method to a substrate,
d) culturing the cell preparation applied in step c) of the method,
e) isolating the three-dimensional sweat gland equivalent obtained in step d) of the method.

### C)

A method for the in-vitro investigation of sweating processes, in which

a three-dimensional sweat gland equivalent is provided,

the three-dimensional sweat gland equivalent is stimulated adrenergically,

the three-dimensional sweat gland equivalent is brought into contact with an active substance to be investigated, and

the reaction of the three-dimensional sweat gland equivalent is determined by measuring the cyclic adenosine monophosphate (cAMP) content,

wherein the three-dimensional sweat gland equivalent comprises from about 500 to about 500000 sweat gland cells and/or has a diameter of from about 100 μm to about 6000 μm, preferably of from about 150 μm to about 4000 μm, more preferably of from about 200 μm to about 3000 μm.

### D)

A method for the in-vitro investigation of sweating processes, in which

a three-dimensional sweat gland equivalent is provided,

the three-dimensional sweat gland equivalent is stimulated adrenergically,

the three-dimensional sweat gland equivalent is brought into contact with an active substance to be investigated, and

the reaction of the three-dimensional sweat gland equivalent is determined by colorimetric measurement of the cyclic adenosine monophosphate (cAMP) content, wherein the three-dimensional sweat gland equivalent comprises from about 500 to about 500000 sweat gland cells and/or has a diameter of from about 100 μm to about 6000 μm, preferably of from about 150 μm to about 4000 μm, more preferably of from about 200 μm to about 3000 μm.

### E)

A method for the in-vitro investigation of sweating processes, in which

a three-dimensional sweat gland equivalent is provided,

the three-dimensional sweat gland equivalent is stimulated adrenergically,

the three-dimensional sweat gland equivalent is brought into contact with an active substance to be investigated, and

the reaction of the three-dimensional sweat gland equivalent is determined by colorimetric measurement of the cyclic adenosine monophosphate (cAMP) content, wherein the three-dimensional sweat gland equivalent comprises from about 500 to about 500000 sweat gland cells and/or has a diameter of from about 100 μm to about 6000 μm, from about 150 μm to about 4000 μm, or from about 200 μm to about 3000 μm, and wherein the three-dimensional sweat cell equivalents are produced from eccrine sweat glands.

The embodiments A to E may be combined with other preferred embodiments. With the aid of the 3D sweat gland model, active substances which could be used as antiperspirants and so could replace conventional aluminum-containing products can be identified when screening active substances using the method as contemplated herein.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A method for the in-vitro investigation of sweating processes, in which
a three-dimensional sweat gland equivalent is provided,
the three-dimensional sweat gland equivalent is stimulated adrenergically, and
the reaction of the three-dimensional sweat gland equivalent is determined by measuring the cyclic adenosine monophosphate (cAMP) content.

2. The method as claimed in claim 1, wherein the three-dimensional sweat gland equivalent comprises from about 500 to about 500000 sweat gland cells and/or has a diameter of from about 100 μm to about 6000 μm.

3. The method of claim 2, wherein the three dimensional sweat gland equivalent has a diameter of from about 150 μm to about 4000 μm.

4. The method of claim 2, wherein the three dimensional sweat gland equivalent has a diameter of from about 200 μm to about 3000 μm.

5. The method as claimed in claim 1, wherein the three-dimensional sweat gland equivalent is present in an aqueous medium or is dispersed in water.

6. The method as claimed in claim 1, wherein the three-dimensional sweat gland equivalent is produced by means of a method that comprises the following steps:
a) providing isolated sweat glands,
b) providing a cell preparation of primary sweat gland cells from the sweat glands isolated in step a) of the method,
c) applying the cell preparation provided in step b) of the method to a substrate,
d) culturing the cell preparation applied in step c) of the method,
e) isolating the three-dimensional sweat gland equivalent obtained in step d) of the method.

7. The method as claimed in claim 6, wherein the sweat glands provided in step a) of the method are human eccrine and/or apocrine sweat glands.

8. The method as claimed in claim 6, wherein the primary sweat gland cells provided in step b) of the method comprise at least one cell type selected from the group formed by (i) coil cells, (ii) duct cells, as well as (iii) mixtures thereof.

9. The method as claimed in claim 1, wherein the three-dimensional sweat gland equivalent is stimulated, wherein adrenaline, is added to the three-dimensional sweat gland equivalent.

10. The method of claim 1, wherein the three-dimensional sweat gland equivalent is stimulated by an adrenergic drug.

11. The method as claimed in claim 10, wherein the adrenergic drug is an adrenergic receptor agonist and/or an adrenergic reuptake inhibitor.

12. The method as claimed in claim 10, wherein the adrenergic drug is adrenaline.

13. The method as claimed in claim 1, wherein the reaction of the three-dimensional sweat gland equivalent is determined by colorimetric measurement of the cyclic adenosine monophosphate (cAMP) content.

14. The method as claimed in claim 1, wherein the reaction of the three-dimensional sweat gland equivalent to an active substance which is envisaged as reducing the sweating process is determined on the three-dimensional sweat gland equivalent.

15. The method as claimed in claim 14, wherein the reaction of the three-dimensional sweat gland equivalent to the active substance is determined, wherein the active substance is added to the three-dimensional sweat gland equivalent, or at the same time as the step of the method in which the three-dimensional sweat gland equivalent is stimulated, or after the three-dimensional sweat gland equivalent has been stimulated.

16. A method for screening active substances for antiperspirant activity, wherein
a three-dimensional sweat gland equivalent is provided,
the three-dimensional sweat gland equivalent is stimulated adrenergically,
the three-dimensional sweat gland equivalent is brought into contact with an active substance to be investigated before, during and/or after adrenergic stimulation, and
the reaction of the three-dimensional sweat gland equivalent is determined by measuring the cyclic adenosine monophosphate (cAMP) content, wherein a reduction in cAMP content compared to a control that has not been brought into contact with an active substance is indicative of antiperspirant activity.
